(19) United States
(12) Patent Application Publication
(10) Pub. No.: US 2013/0190268 A1
(43) Pub. Date: Jul. 25, 2013

(54) BIOTINYLATED POLYSACCHARIDES HAVING AN ANTITHROMBOTIC ACTIVITY AND IMPROVED METABOLIC STABILITY

(71) Applicant: SANOFI, Paris (FR)

(72) Inventors: Marc BIANCIOTTO, Pinsague (FR); Pierre Alexandre DRIGUEZ, Le Mesnil Le Roi (FR); Phillippe DUCHAUSSOY, Toulouse (FR); Gilbert LASSALLE, Les Molieres (FR); Pierrick LE-DREF, Volonne (FR); Jean-Christophe THIERY, Marat (FR); Patrick TROUILLEUX, SISTERON (FR)

(73) Assignee: SANOFI, Paris (FR)

(21) Appl. No.: 13/790,681

(22) Filed: Mar. 8, 2013

Related U.S. Application Data
(63) Continuation of application No. PCT/FR2011/000498, filed on Sep. 9, 2011.

(30) Foreign Application Priority Data
Sep. 10, 2010 (FR) 1057194
May 30, 2011 (FR) 1154709

(51) Int. Cl. C07H 15/26

(52) U.S. Cl. CPC C07H 15/26
USPC 514/54; 536/53

(57) ABSTRACT

The invention relates to novel polysaccharides with an antithrombotic activity, having at least one covalent bond with biotin or a biotin derivative, wherein said covalent bond is resistant to metabolic cleavage and comprises a linkage X selected from the group consisting of —O—, —N(R)—, —N(R)—CO— and —N(R′)—CO—N(R″)—, wherein R is an alkyl group and R′ and R″, which may be identical or different, are, independently of one another, a hydrogen atom or alkyl group.

The present invention relates to novel synthetic oligosaccharides and polysaccharides having at least one covalent bond with biotin or a biotin derivative and having the anticoagulant and antithrombotic pharmacological activities of heparin.

Patent application WO 02/24754 describes synthetic polysaccharides which have a covalent bond with biotin (hexahydro-2-oxo-1H-thieno[3,4-d]imidazole-4-pentanoic acid) or with a biotin derivative. Such polysaccharides have an antithrombotic activity which means that they can be used as anticoagulants, and also have the advantage of being able to be rapidly neutralized with a specific antidote, in an emergency situation. This specific antidote is avidin (The Merck Index, Twelfth edition, 1996, M.N. 920, pages 151-152) or streptavidin, two tetrameric proteins of respective weights equal to approximately 66 000 and 60 000 Da, which have a very strong affinity for biotin.

Patent application WO 02/24754 describes in particular the following compound, known as idrabiotaparinux:

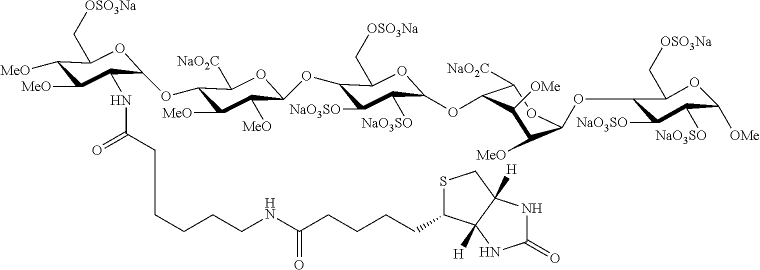

In the mammalian body, idrabiotaparinux is partly metabolized at the level of the amide bond adjacent to the biotin group, thus producing a pentasaccharide compound bearing an amine chain —NH—CO—(CH₂)₅—NH₂ on the first glucosamine unit, as described in patent application WO 2010/023374.

It may be desirable, in particular in the context of clinical developments of molecules of pharmaceutical interest, to limit or even prevent the metabolization of compounds of this type.

Novel polysaccharides with structures analogous to some of those described in patent application WO 02/24754 have now been identified, which polysaccharides have antithrombotic properties and a neutralization capacity, for example via avidin, which are comparable to those described in that patent application, but which also have improved metabolic stability.

Generally, the invention therefore relates to synthetic polysaccharides with antithrombotic activity having at least one covalent bond with biotin or a biotin derivative, characterized in that said covalent bond is resistant to metabolic cleavage and comprises a linkage X chosen from —O—, —N(R)—, —N(R)—CO— and —N(R′)—CO—N(R″)—, in which R is an alkyl group and R′ and R″, which may be identical or different, are, independently of one another, hydrogen atoms or alkyl groups.

For the purposes of the present invention, and unless otherwise mentioned in the text, the term “alkyl” is intended to mean a linear or branched, saturated aliphatic group comprising from 1 to 6 carbon atoms, and advantageously a methyl group.

Biotin, or hexahydro-2-oxo-1H-thieno[3,4-d]imidazole-4-pentanoic acid, is the compound having the following formula:

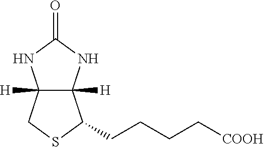 [SMILES: O=C(O)CCCC[C@@H]1SC[C@@H]2NC(=O)N[C@@H]21]

By way of biotin derivatives, mention may be made of those indicated in the Pierce catalog 1999-2000, pages 62 to 81, or in patent application WO 02/24754.

More particularly, the invention relates to synthetic polysaccharides with antithrombotic activity having at least one covalent bond with biotin or a biotin derivative, characterized in that said covalent bond is a linkage of formula T hereinafter:

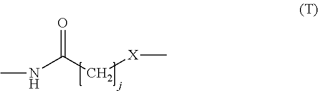

in which j is an integer which can take any value from 1 to 10 and X is chosen from the linkages —O—, —N(R)—, —N(R)—CO— and —N(R′)—CO—N(R″)—, in which R is an alkyl group and R′ and R″, which may be identical or different, are, independently of one another, hydrogen atoms or alkyl groups.

Even more particularly, the invention relates to synthetic polysaccharides with antithrombotic activity having at least one covalent bond with biotin or a biotin derivative, characterized in that said covalent bond with biotin or the biotin derivative corresponds to the formula below:

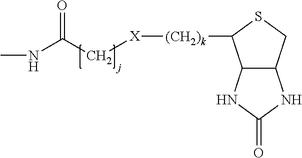

in which j is an integer which can take any value from 1 to 10, k is an integer which can take any value from 4 to 10, and X is chosen from the linkages —O—, —N(R)—, —N(R)—CO— and —N(R′)—CO—N(R″)—, in which R is an alkyl group and R′ and R″, which may be identical or different, are, independently of one another, hydrogen atoms or alkyl groups.

According to one advantageous embodiment of the invention, said polysaccharides are 5 sugars long, that is to say they are pentasaccharides.

Generally, a subject of the present invention is the polysaccharides of formula (I):

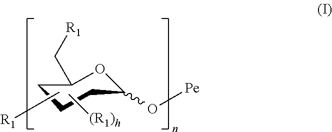

in which:
the wavy line denotes a bond located either above or below the plane of the pyranose ring,
the formula:

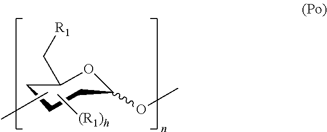

denotes a polysaccharide containing n identical or different monosaccharide units, bonded via its anomeric carbon to Pe,
the formula:

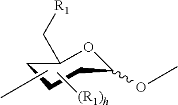

is a diagrammatic representation of a monosaccharide unit with a pyranose structure, chosen from hexoses, pentoses and the corresponding deoxy sugars, this unit being bonded via its anomeric carbon to another monosaccharide unit, and the hydroxyl groups of this unit being substituted with identical or different R₁ groups, R₁ being as defined below,
Pe is a pentasaccharide having the structure:

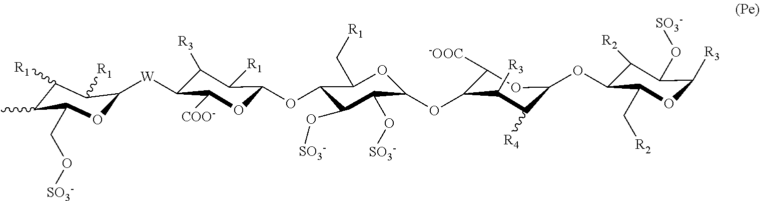

h is equal to 1 or 2,
n is an integer and can take any value from 0 to 25,
R₁ is the linkage -T-Biot, a (C₁-C₆)alkoxy group or an —OSO₃⁻ group,
R₂ is the linkage -T-Biot, a (C₁-C₆)alkoxy group or an —OSO₃⁻ group,
R₃ is the linkage -T-Biot or a (C₁-C₆)alkoxy group,
R₄ is the linkage -T-Biot, a (C₁-C₆)alkoxy group or an —OSO₃⁻ group or else R₄ constitutes an —O—CH₂— bridge, the —CH₂— group being bonded to the carbon atom bearing the carboxylic function on the same ring;
   it being understood that at least one of the substituents R₁, R₂, R₃ or R₄ is the linkage -T-Biot,
W is an oxygen atom or a methylene group,
T is the linkage:

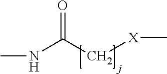

in which j is an integer which can take any value from 1 to 10 and X is chosen from the linkages —O—, —N(R)—, —N(R)—CO— and —N(R′)—CO—N(R″)—, in which R is an alkyl group and R′ and R″, which may be identical or different, are, independently of one another, hydrogen atoms or alkyl groups,
Biot is the group:

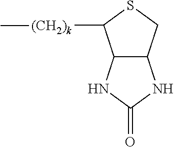

in which k is an integer which can take any value from 4 to 10;
and also the pharmaceutically acceptable salts thereof.

Such polysaccharides according to the present invention have improved metabolic stability compared with the polysaccharides in which T is the linkage —NH—CO—(CH₂)ⱼ—NH—CO—, in which j is an integer which can take any value from 1 to 10, such as those described in patent application WO 02/24754.

As previously indicated, it will be noted that, in general, in the present description, a wavy line denotes a bond located either below or above the plane of the pyranose ring.

The monosaccharides contained in Po may be identical to or different than one another, and the interglycosidic bonds may be of α or β type.

These monosaccharides are advantageously chosen from the D or L hexoses alose, altrose, glucose, mannose, galose, idose, galactose and talose (in this case, h=2) or from the D or L pentoses ribose, arabinose, xylose and lyxose (in this case, h=2).

Other monosaccharides, such as, for example, deoxy sugars, can also be used (h=1 and/or —CH₂R₁═CH₃).

The polysaccharide part Po can consist of from 0 to 25 alkylated and di- or trisulfated monosaccharide units.

The polysaccharide part Po can also consist of from 0 to 25 alkylated and mono- or disulfated monosaccharide units.

The polysaccharide part Po can consist of from 0 to 25 uncharged and/or partially charged and/or totally charged alkylated monosaccharide units.

The charged or uncharged units can be dispersed all along the chain or they can, on the contrary, be grouped into charged or uncharged saccharide domains.

The bonds between the units may be 1,2; 1,3; 1,4; 1,5; 1,6; and of α or β type.

In the present description, it has been chosen to represent the conformations ¹C₄ for L-iduronic acid and ⁴C₁ for D-glucuronic acid, but it is well known that, generally, the conformation in solution of the monosaccharide units fluctuates. Thus, L-iduronic acid may be of ¹C₄, ²S₀ or ⁴C₁ conformation.

According to one of its aspects, the invention relates to the polysaccharides of formula (I.1).

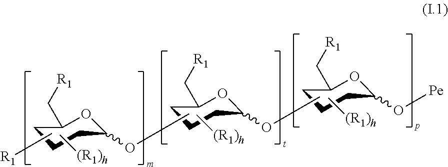

in which:
the formula:

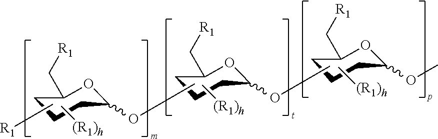

denotes a particular family of polysaccharides Po, bonded via their anomeric carbon to Pe as defined for (I),
the formula:

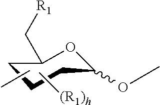

is as defined for (I),
the R₁ groups are as defined for (I) and, for one and the same monosaccharide, may be identical or different,
the monosaccharide contained in [ ]ₘ is repeated m times, the monosaccharide contained in [ ]ₜ is repeated t times, the monosaccharide contained in [ ]ₚ is repeated p times,
m is an integer ranging from 1 to 5, t is an integer ranging from 0 to 24 and p is an integer ranging from 0 to 24, it being understood that 1≦m+t+p≦25,
   and also the pharmaceutically acceptable salts thereof.

Among these polysaccharides of formula (I.1), the polysaccharides in which just one of the substituents R₁, R₂, R₃ or R₄ is the linkage T-Biot with T and Biot being as defined for formula (I), and also the pharmaceutically acceptable salts thereof, constitute a subgroup of the invention.

According to another of its aspects, the invention relates to the pentasaccharides of formula (I.2):

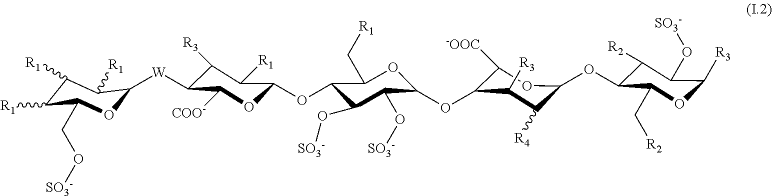

in which R₁, R₂, R₃, R₄ and W are as defined for formula (I), and also the pharmaceutically acceptable salts thereof.

Among these pentasaccharides of formula (I.2), the pentasaccharides in which just one of the substituents R₁, R₂, R₃ or R₄ is a linkage T-Biot, with T and Biot being as defined for formula (I), and also the pharmaceutically acceptable salts thereof, constitute another subgroup of the invention.

Among these pentasaccharides of formula (I.2), a subject of the invention is also the pentasaccharides of formula (I.3):

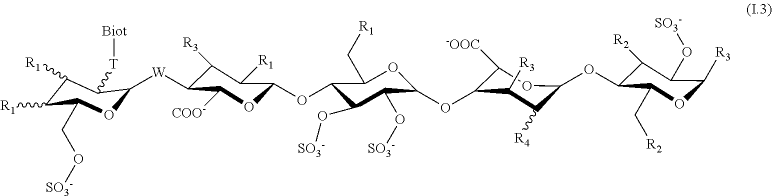

in which:
T and Biot are as previously defined,
R₁ is a (C₁-C₆)alkoxy group or an —OSO₃⁻ group,
R₂ is a (C₁-C₆)alkoxy group or an —OSO₃⁻ group,
R₃ is a (C₁-C₆)alkoxy group,
R₄ is a (C₁-C₆)alkoxy group or an —OSO₃⁻ group, or else R₄ constitutes an —O—CH₂— bridge, the —CH₂— group being bonded to the carbon atom bearing the carboxylic function on the same ring,
W is an oxygen atom or a methylene group,
   and also the pharmaceutically acceptable salts thereof.

Among the compounds of the invention, one subgroup of compounds is such that the index j (present in the linkage T) is equal to 4 or 5.

Another subgroup of compounds is such that the index k (present in the Biot group) is equal to 4 or 5.

A subject of the invention is more particularly the compounds previously defined in which X is chosen from the linkages —O—, —N(R)— and —N(R′)—CO—N(R″)—, in which R is an alkyl group and R′ and R″, which may be identical or different, are, independently of one another, hydrogen atoms or alkyl groups.

The structure of such compounds is particularly original in that it no longer comprises a biotin group as such. This is because the carbonyl group naturally present at the level of the alkylene arm of native biotin (pentanoic acid end group), which group is present in the idrabiotaparainux compound at the level of the amide bond providing bonding between the biotin and the pentasaccharide, via the alkylene linker, is absent in these compounds according to the invention. Despite this structural alteration, the compounds according to the invention nevertheless retain the capacity to be neutralized with avidin, as will subsequently be demonstrated.

According to another of its aspects, the invention relates to the following pentasaccharides:
Methyl (2-deoxy-3,4-di-O-methyl-2-{[6-({5-[(3aS,4S,6aR)-2-oxohexahydro-1H-thieno[3,4-d]imidazol-4-yl]pentyl}oxy)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt (compound No. 1);
Methyl (2-deoxy-3,4-di-O-methyl-2-{[6-(methyl{5-[(3aS,4S,6aR)-2-oxohexahydro-1H-thieno[3,4-d]imidazol-4-yl]pentyl}amino)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt (compound No. 2);
Methyl (2-deoxy-3,4-di-O-methyl-2-{[6-(methyl{5-[(3aS,4S,6aR)-2-oxohexahydro-1H-thieno[3,4-d]imidazol-4-yl]pentanoyl}amino)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt (compound No. 3);
Methyl (2-deoxy-3,4-di-O-methyl-2-({5-[({4-[(3aS,4S,6aR)-2-oxohexahydro-1H-thieno[3,4-d]imidazol-4-yl]butyl}carbamoyl)amino]pentanoyl}amino)-6-O-sulfonato-α-D-glucopyranosyl)-(1→4)-(2,3-di-O-methyl-β-D-glucopyranosyluronate)-(1→4)-(2,3,6-tri-β-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt (compound No. 4).

The invention encompasses the polysaccharides in the acid form thereof or in the form of any one of the pharmaceutically acceptable salts thereof. In the acid form, the —COO⁻ and —SO₃⁻ functions are respectively in —COOH and —SO₃H form.

The expression “pharmaceutically acceptable salt of the polysaccharides of the invention” is intended to mean a polysaccharide in which one or more of the —COO⁻ and/or —SO₃⁻ functions are ionically bonded to a pharmaceutically acceptable cation. The salts which are preferred according to the invention are those of which the cation is chosen from alkaline metal cations and even more preferably those of which the cation is Na⁺ or K⁺.

In its principle, the process for preparing the compounds according to the invention uses disaccharide or oligosaccharide basic synthons prepared as previously reported in the literature. Reference will in particular be made to the patents or patent applications EP 0 300 099, EP 0 529 715, EP 0 621 282 and EP 0 649 854 and also to the document C. van Boeckel, M. Petitou, Angew. Chem. Int. Ed. Engl., 1993, 32, 1671-1690. These synthons are subsequently coupled to one another so as to provide an entirely protected equivalent of a polysaccharide according to the invention. This protected equivalent is subsequently converted into a compound according to the invention.

One of the basic synthons mentioned above contains a particular protected function which enables the subsequent introduction of the biotin or of the biotin derivative, for example a latent amine function in the form of an azido group or protected in the form of N-phthalimido.

In the coupling reactions mentioned above, a “donor” disaccharide or oligosaccharide, activated on its anomeric carbon, reacts with an “acceptor” disaccharide or oligosaccharide which has a free hydroxyl.

The present invention relates to a process for preparing the compounds of formula (I), characterized in that: in a first step, a completely protected equivalent of the desired polysaccharide (I), containing a protected precursor of the Pe domain extended at its nonreducing end by a protected precursor of the sulfated polysaccharide Po, is synthesized, one of these precursors containing in particular an amine function which is suitably protected for the subsequent introduction of the biotin or the biotin derivative; in a second step, the negatively charged groups are introduced and/or unmasked; in a third step, the amine function on the polysaccharide is deprotected and then the biotin or the biotin derivative is introduced.

The synthesis of Pe is carried out according to the methods described in particular in the patent applications published under numbers WO 98/03554 and WO 99/36443, and also in the literature on polysaccharides.

The synthesis of the Po precursor polysaccharide part is carried out according to reactions well known to those skilled in the art, using the oligosaccharide synthesis methods (G. J. Boons, Tetrahedron, 1996, 52, 1095-1121, WO 98/03554 and WO 99/36443). Typically, an oligosaccharide which is a glycosidic bond donor is coupled with an oligosaccharide which is a glycosidic bond acceptor so as to give another oligosaccharide, the size of which is equal to the sum of the sizes of the two reactive species. This sequence is repeated until the desired compound of formula (I) is obtained. The nature and the profile of the charge of the desired final compound determine the nature of the chemical entities used in the various steps of the synthesis, according to the rules well known to those skilled in the art. Reference may, for example, be made to C. van Boeckel, M. Petitou, Angew. Chem. Int. Ed. Engl., 1993, 32, 1671-1690 or else to H. Paulsen, “Advances in selective chemical syntheses of complex oligosaccharides” Angew. Chem. Int. Ed. Engl., 21, 155-173 (1982).

The compounds of the invention are obtained from their completely protected polysaccharide precursors using the following series of reactions:
the alcohol functions which have to be converted into an O-sulfo group and the carboxylic acids are deprotected by removing the protective groups used during the production of the backbone,
the sulfo groups are then introduced,
the amine function of the polysaccharide making it possible to introduce the biotin or the biotin derivative is deprotected,
the biotin or the biotin derivative is introduced via a conventional amino/acid coupling reaction.

The compounds of the invention can naturally be prepared by using various strategies known to those skilled in the art for the synthesis of oligosaccharides.

The process described above is the preferred process of the invention. However, the compounds of formula (I) can be prepared by other well-known methods of sugar chemistry described, for example, in “Monosaccharides, Their chemistry and their roles in natural products”, P. M. Collins and R. J. Ferrier, J. Wiley & Sons, 1995 and in G. J. Boons, Tetrahedron, 1996, 52, 1095-1121.

The pentasaccharides Pe can therefore be obtained from disaccharide synthons in the manner described in the publication by C. van Boeckel, M. Petitou, Angew. Chem. Int. Ed. Engl., 1993, 32, 1671-1690.

In general, the protective groups used in the process for preparing the compounds (I) are those commonly used in sugar chemistry, as described, for example, in Protective Groups in Organic Synthesis, T W Greene, John Wiley & Sons, New York, 1981. The protective groups are advantageously chosen, for example, from acetyl, halomethyl, benzoyl, levulinyl, benzyl, substituted benzyl, optionally substituted trityl, tetrahydropyranyl, allyl, pentenyl, tert-butyldimethylsilyl (tBDMS) or trimethylsilylethyl groups.

The activating groups are those conventionally used in sugar chemistry according to, for example, G. J. Boons, Tetrahedron, 1996, 52, 1095-1121. These activating groups are chosen, for example, from imidates, thioglycosides, pentenylglycosides, xanthates, phosphites or halides.

As regards the way in which the biotin is bonded to the oligosaccharide and also the nature of the biotin derivative, the chemical literature offers other possibilities that can be implemented using sets of protective groups well known to those skilled in the art. Use will preferably be made of an amine function, or else a thiol function, or else a carboxylic acid function or else an aldehyde function, that will be reacted with a biotin derivative comprising a reactive group of the activated ester, maleimide, iodoacetyl or primary amine type, the reaction being carried out according to the conditions described in the literature (cf. Savage et al., Avidin-Biotin Chemistry: A Handbook; Pierce Chemical Company, 1992).

The process described above makes it possible to obtain the compounds of the invention in the form of salts. To obtain the corresponding acids, the compounds of the invention in the form of salts are brought into contact with a cation exchange resin in acid form. The compounds of the invention in the form of acids can subsequently be neutralized with a base so as to obtain the desired salt. Any inorganic or organic base which gives, with the compounds of formula (I), pharmaceutically acceptable salts can be used to prepare the salts of the compounds of formula (I). Sodium hydroxide, potassium hydroxide, calcium hydroxide or magnesium hydroxide is preferentially used as base. The sodium and calcium salts of the compounds of formula (I) are the preferred salts.

The invention will be understood more clearly from the detailed examples which follow, relating to the preparation of compounds according to the invention. These examples are not limiting and merely illustrate the present invention.

The starting compounds and the reagents, when the method for preparing them is not expressly described, are commercially available or described in the literature, or else can be prepared according to methods which are described therein or which are known to those skilled in the art. The following abbreviations are used:

[α]_{D}: optical rotation

### ESI: ElectroSpray Ionization

h: hour
LC-MS: liquid chromatography coupled to mass spectrometry
Me: methyl
min: minutes
ml: milliliter
TFA: trifluoroacetic acid
T_{R}: retention time measured by LC-MS

The LC-MS operations are carried out on a Waters ZQ4000 apparatus. The column used is a Symmetry C18 3.5 μm (2.1×50 mm). The eluent A consists of H₂O+0.005% TFA, pH 3.15. The eluent B consists of acetonitrile+0.005% TFA. The gradient ranges from 0 to 90% of eluent B in 10 (or 30) min+5 min at 90% of eluent B. The flow rate is 0.4 ml/min.

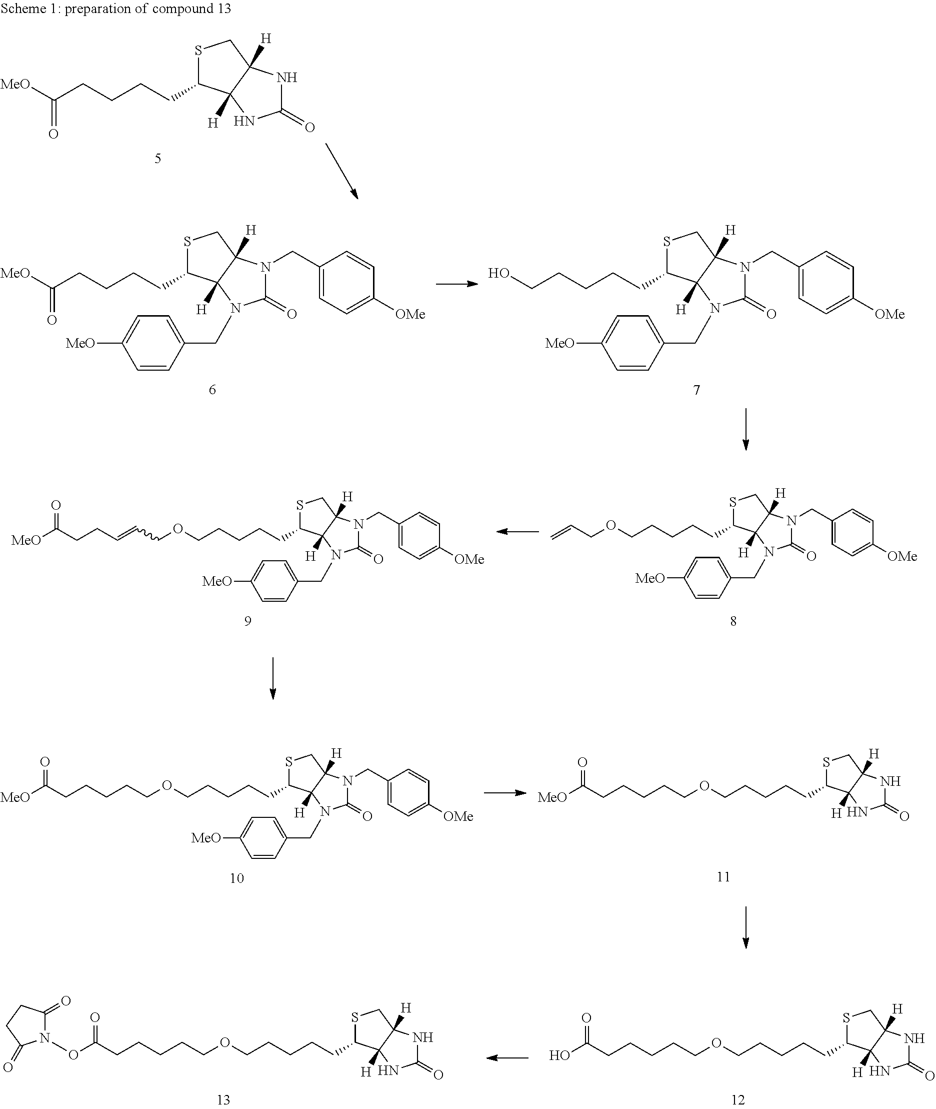

### Preparation of methyl 5-[(3aS,4S,6aR)-1,3-bis(4-methoxybenzyl)-2-oxohexahydro-1H-thieno[3,4-d]imidazol-4-yl]pentanoate (6)

Sodium hydride (0.613 g, 15.3 mmol) and then p-methoxybenzyl chloride (2.9 ml, 20.9 mmol) are added successively, at 0° C., to a solution of methyl ester of D(+)-biotin or methyl 5-[(3aS,4S,6aR)-2-oxohexahydro-1H-thieno[3,4-d]imidazol-4-yl]pentanoate 5 (1.80 g, 6.97 mmol) (prepared according to the method described in Tetrahedron Letters 1989, 30, 3089-3092) in N,N-dimethylformamide (42 ml). After stirring for 4 h at 0° C., methanol (1.5 ml) is added. After stirring for 30 min, the reaction medium is diluted with ethyl acetate (400 ml). The organic phase is washed with water, dried over sodium sulfate, filtered and then evaporated to dryness. The resulting residue is used directly in the next reaction.

LC-MS m/z 499.2 [(M+H)⁺]. T_{R}=9.157 min

### Preparation of (3aS,4S,6aR)-4-(5-hydroxypentyl)-1,3-bis(4-methoxybenzyl)tetrahydro-1H-thieno[3,4-d]imidazol-2(3H)-one (7)

The crude compound 6 (6.97 mmol) is dissolved in tetrahydrofuran (140 ml) and then, at 0° C., a 1M solution of LiAlH₄ in tetrahydrofuran (7 ml) is added. After stirring for 1 h 30, the excess LiAlH₄ is destroyed by addition, at 0° C., of tetrahydrofuran containing 5% of water (30 ml) and then of tert-butanol (30 ml), and the complex is decomposed with a saturated aqueous solution of sodium sulfate (25 ml). The solvent is evaporated off and then the residue obtained is diluted with dichloromethane (400 ml). The organic phase is washed with water, dried over sodium sulfate, filtered and then evaporated to dryness. The residue obtained is purified by flash chromatography on a silica gel column (66/34 toluene/acetone) to give the compound 7 (3.05 g, 93% over the 2 stages).

LC-MS m/z 471.2 [(M+H)⁺]. T_{R}=8.251 min

### Preparation of (3aS,4S,6aR)-1,3-bis(4-methoxybenzyl)-4-[5-(prop-2-en-1-yloxy)pentyl]tetrahydro-1H-thieno[3,4-d]imidazol-2(3H)-one (8)

Sodium hydride (1.30 g, 32.4 mmol) and 3-iodopropene (2.4 ml, 26 mmol) are added successively, at 0° C., to a solution of the compound 7 (3.05 g, 6.48 mmol). After stirring for 5 h at 0° C., methanol (2.7 ml) is added. After stirring for 30 min, the reaction medium is concentrated to dryness and then taken up with ethyl acetate (450 ml). The organic phase is washed with water, dried over sodium sulfate, filtered and then evaporated to dryness. The residue obtained is purified by flash chromatography on a silica gel column (85/15 toluene/acetone) to give the compound 8 (3.02 g, 91%).

LC-MS m/z 511.3 [(M+H)⁺]. T_{R}=1.670 min

### Preparation of methyl (4E,Z)-6-({5-[(3aS,4S,6aR)-1,3-bis(4-methoxybenzyl)-2-oxohexahydro-1H-thieno[3,4-d]imidazol-4-yl]pentyl}oxy)hex-4-enoate (9)

A solution of the compound 8 (2.16 g, 4.23 mmol), of methyl 4-pentanoate (1.05 ml, 8.47 mmol) and of the Grubbs catalyst (177 mg, 0.21 mmol) in dichloromethane (42 ml) is heated at 45° C. for 16 h. After concentration of the reaction medium, the residue obtained is purified by flash chromatography on a silica gel column (36/64 toluene/ethyl acetate) to give the compound 9 (675 mg, 27%).

LC-MS m/z 597.5 [(M+H)⁺]. T_{R}=1.654 min

### Preparation of methyl 6-({5-[(3aS,4S,6aR)-1,3-bis(4-methoxybenzyl)-2-oxohexahydro-1H-thieno[3,4-d]imidazol-4-yl]pentyl}oxy)hexanoate (10)

A solution of the compound 9 (915 mg, 1.53 mmol) in a mixture of dichloromethane (77 ml) and tert-butanol (77 ml) is stirred under a hydrogen atmosphere (5 bar) in the presence of Pd/C 10% (2.3 g) for 4 h. After filtration, the medium is concentrated to dryness. The residue obtained is purified by flash chromatography on a silica gel column (3/2 toluene/ethyl acetate) to give the compound 10 (756.8 mg, 82%).

LC-MS m/z 599.4 [(M+H)⁺]. T_{R}=1.71 min

### Preparation of methyl 6-({5-[(3aS,4S,6aR)-2-oxohexahydro-1H-thieno[3,4-d]imidazol-4-yl]pentyl}oxy)hexanoate (11)

The compound 10 (358 mg, 0.6 mmol) is dissolved in trifluoroacetic acid (15 ml, 25 ml/mmol). After stirring for 16 h at ambient temperature, the reaction medium is codistilled with toluene (3×50 ml) and then concentrated to dryness. The residue obtained is purified by flash chromatography on a silica gel column (9/1 dichloromethane/methanol) to give the compound 11 (176 mg, 82%).

LC-MS m/z 359.2 [(M+H)⁺]. T_{R}=1.085 min

### Preparation of 6-({5-[(3aS,4S,6aR)-2-oxohexahydro-1H-thieno[3,4-d]imidazol-4-yl]pentyl}oxy)hexanoic acid (12)

A solution of the compound 11 (170 mg, 0.47 mmol) in dioxane (6 ml, 12 ml/mmol) is heated at 95° C. in the presence of an aqueous 1M solution of hydrochloric acid (6 ml, 12 ml/mmol) for 16 h. The reaction mixture is concentrated to dryness and then codistilled with toluene (3×10 ml). The resulting residue is used directly in the next reaction.

LC-MS m/z 345.2 [(M+H)⁺]. T_{R}=0.904 min

### Preparation of 1{[6-({5-[(3aS,4S,6aR)-2-oxohexahydro-1H-thieno[3,4-d]imidazol-4-yl]pentyl}oxy)hexanoyl]oxy}pyrrolidine-2,5-dione (13)

Triethylamine (147 μl, 1.05 mmol) and ethyl chloroformate (99.5 μl, 1.05 mmol) are added successively, at 0° C., to a suspension of the compound 12 (60 mg, 0.17 mmol) in N,N-dimethylformamide (3.5 ml). After stirring for 1 h at 0° C., N-hydroxysuccinimide (120 mg, 1.05 mmol) is added. After stirring for 16 h at ambient temperature, the reaction medium is concentrated. The residue obtained is purified by flash chromatography on a silica gel column (6/1 dichloromethane/methanol) to give the compound 13 (66.7 mg, 86%).

LC-MS m/z 442.2 [(M+H)⁺]. T_{R}=6.904 min

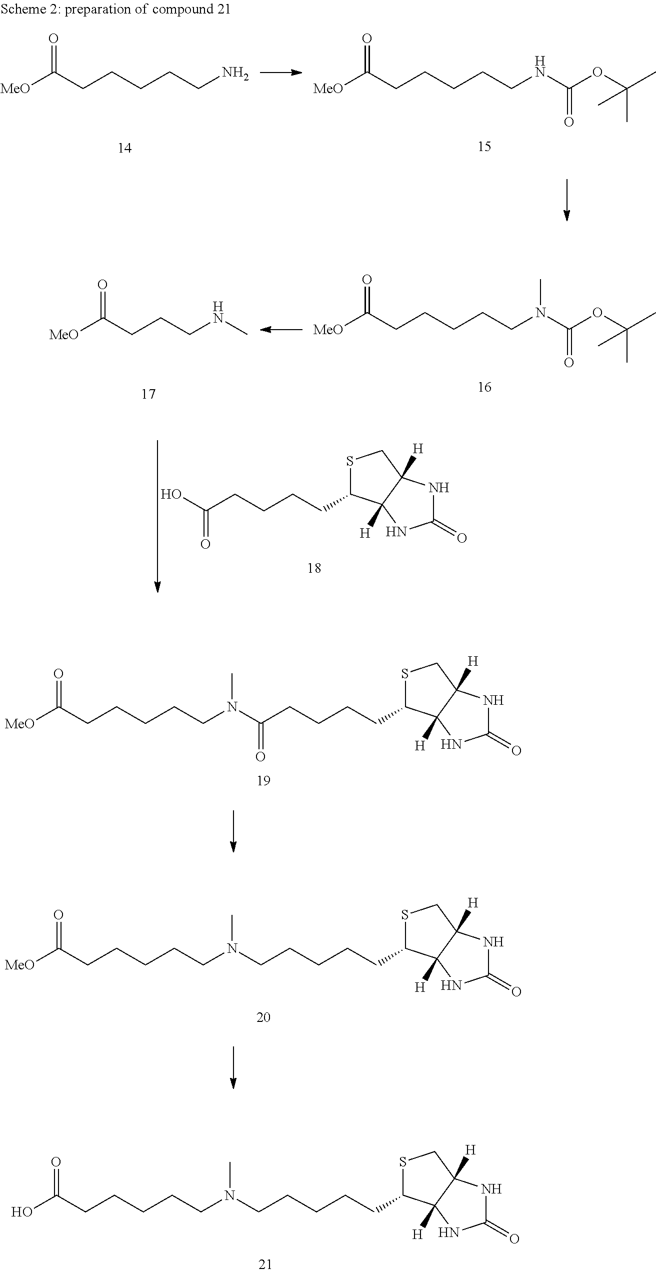

### Preparation of methyl 6-[(tert-butoxycarbonyl)amino]hexanoate (15)

Trimethylamine (30.6 ml, 220 mmol) is added to a solution of methyl 6-aminocaproate 14 (20.0 g, 110 mmol) in dichloromethane (50 ml), and then a solution of di-tert-butyl dicarbonate (26.4 g, 121.1 mmol) in dichloromethane (50 ml) is added at 0° C. over the course of 45 min. After 2 h at 0° C., the reaction mixture is washed with water and then the organic phase is dried (Na₂SO₄), filtered and concentrated. The resulting residue (27 g) is used in the next stage without purification.

[M+H⁺]=246.3

### Preparation of methyl 6-[(tert-butoxycarbonyl)(methyl)amino]hexanoate (16)

A solution of the crude compound 15 (12.0 g) in tetrahydrofuran (60 ml) and then iodomethane (9.15 ml, 146.7 mmol) are added dropwise, over the course of 5 min at ambient temperature, to a suspension of sodium hydride (2.2 g at 60%, 53.8 mmol) in tetrahydrofuran (60 ml). After 1 h at 50° C., a further amount of iodomethane (3.05 ml, 48.9 mmol) is added. This operation is again repeated twice. After having cooled, the reaction mixture is washed with water (240 ml) and then the organic phase is dried (Na₂SO₄), filtered and concentrated. The resulting residue (11.5 g) is used in the next stage without purification.

[M+H⁺]=260.4

### Preparation of methyl 6-(methylamino)hexanoate (17)

A 2M solution of hydrochloric acid in diethyl ether (463 ml) is added to a solution of the crude compound 16 (12.0 g) in dioxane (120 ml). The reaction mixture is stirred for 5 h at ambient temperature and is then concentrated to dryness. The resulting residue is purified by chromatography on a silica gel column (95/5→80/20 dichloromethane/methanol over the course of 40 min) to give the compound 17 (3.91 g).

[M+H⁺]=196.7

### Preparation of methyl 6-(methyl{5-[(3aS,4S,6aR)-2-oxohexahydro-1H-thieno[3,4-d]imidazol-4-yl]pentanoyl}amino)hexanoate (19)

Triethylamine (3.3 ml, 23.7 mmol) and ethyl chloroformate (2.3 ml, 24 mmol) are successively added, at −5° C., to a solution of D-biotin or 5-[(3aS,4S,6aR)-2-oxohexahydro-1H-thieno[3,4-d]imidazol-4-yl]pentanoic acid 18 (4.9 g, 20 mmol) in N,N-dimethylformamide (39 ml). After 1 h30, a solution of the compound 17 (3.9 g, 20 mmol) in N,N-dimethylformamide (20 ml) is added to the reaction mixture. After 3.5 h, the reaction medium is diluted with dichloromethane, washed with an aqueous 0.1 N solution of hydrochloric acid and then with an aqueous solution of sodium hydrogen carbonate at 0.5%, washed with water, dried (Na₂SO₄), filtered and concentrated. The resulting residue is purified by chromatography on a silica gel column (3/2 acetone/ethanol) to give the compound 19 (7.68 g).

[M+H⁺]=386.5

### Preparation of methyl 6-(methyl{5-[(3aS,4S,6aR)-2-oxohexahydro-1H-thieno[3,4-d]imidazol-4-yl]pentyl}amino)hexanoate (20)

The compound 19 (2.5 g, 6.48 mmol) is placed in solution in tetrahydrofuran (125 ml). A 2M solution of borane dimethyl sulfide in tetrahydrofuran (13 ml) is then added. The reaction mixture is brought to reflux for 16 h. After having added methanol (125 ml), the reaction medium is brought to reflux for 2 h and then concentrated to dryness. The resulting residue is purified by chromatography on a C18 silica gel column (1/0→0/1 water/acetonitrile over the course of 45 min) to give the compound 20 (55 mg).

[M+H⁺]=372.6

### Preparation of 6-(methyl{5-[(3aS,4S,6aR)-2-oxohexahydro-1H-thieno[3,4-d]imidaol-4-yl]pentyl}amino)hexanoic acid (21)

An aqueous solution of sodium hydroxide at 35% (88.6 μl, 1.07 mmol) is added, at 20° C., to a solution of the compound 20 (80 mg, 0.21 mmol) in a tetrahydrofuran/water mixture (1/1, 1.6 ml). After stirring for 1 h at 20° C., the reaction mixture is neutralized with acetic acid (49.3 μl, 0.86 mmol) and then concentrated to dryness. The resulting residue is purified by chromatography on a C18 silica gel column (1/0→3/2 water/acetonitrile over the course of 20 min) to give the compound 21 (88 mg).

[M+H⁺]=358.5

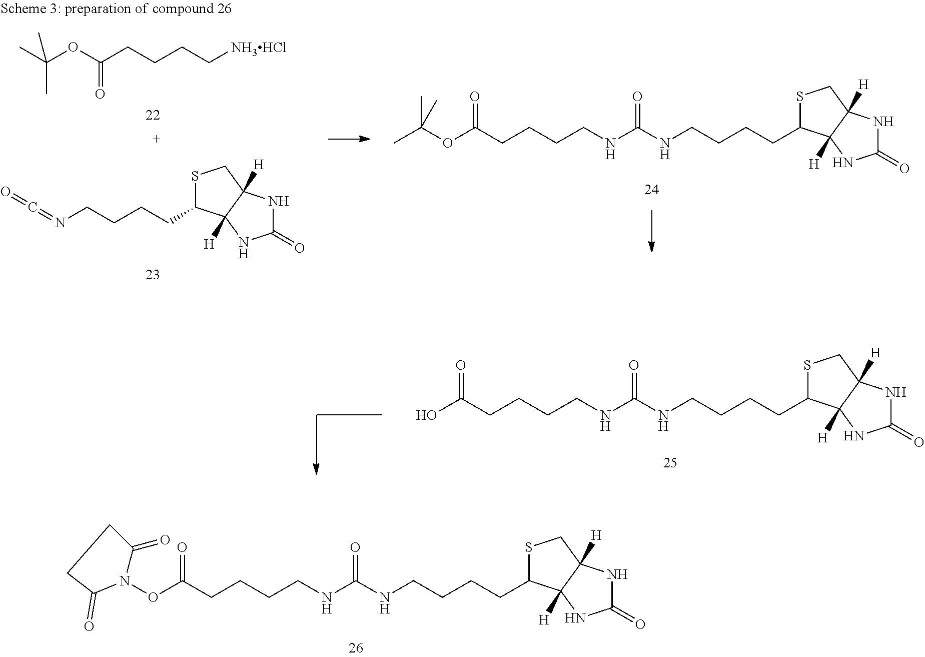

### Preparation of tert-butyl 5-[({4-[(3aS,4S,6aR)-2-oxohexahydro-1H-thieno[3,4-d]imidazol-4-yl]butyl}carbamoyl)amino]pentanoate (24)

A solution of tert-butyl 5-aminopentanoate (22) (640 mg, 3.06 mmol) and triethylamine (458 μl, 3.3 mmol) in a 4:3 mixture of N,N-dimethylformamide and acetonitrile (3.5 ml) is added, at 0° C., to the isocyanate (3aS,4S,6aR)-4-(4-isocyanatobutyl)tetrahydro-1H-thieno[3,4-d]imidazol-2(3H)-one (23) (2.04 mmol) (Murakami, Nobutoshi; Kawanishi, Motoyuki; Itagaki, Sawako; Horii, Toshihiro; Kobayashi, Motomasa; Bioorganic Medicinal Chemistry Letters; 14; 13; 2004; 3513-3516). The temperature of the mixture is left to return to ambient temperature and stirring is maintained for 16 h. The reaction mixture is diluted with water and ethyl acetate and, after extraction of the aqueous phase, the pooled organic phases are dried (Na₂SO₄), filtered and concentrated to give the compound 24 (405 mg). The residue obtained is used in the next stage without purification.

LC-MS m/z 415.4 [(M+H)⁺]. T_{R}=6.497 min

### Preparation of 5-[({4-[(3aS,4S,6aR)-2-oxohexahydro-1H-thieno[3,4-d]imidazol-4-yl]butyl}carbamoyl)amino]pentanoic acid (25)

A solution of the crude compound 24 (396 mg) in formic acid (11.5 ml) is kept under vigorous magnetic stirring at ambient temperature for 5.5 h. The reaction mixture is then concentrated under a high vacuum and then codistilled with toluene and then with acetone until a solid corresponding to the compound 25 is obtained (395 mg). The residue obtained is used in the next stage without purification.

LC-MS m/z 359.1 [(M+H)⁺]. T_{R}=4.848 min

### Preparation of 1-{5-[(2,5-dioxopyrrolidin-1-yl)oxy]-5-oxopentyl}-3-{4-[(3aS,4S,6aR)-2-oxohexahydro-1H-thieno[3,4-d]imidazol-4-yl]butyl}urea (26)

Triethylamine (70 μl, 0.502 mmol) and ethyl chloroformate (48 μl, 0.502 mmol) are added, at 0° C., to a solution of the crude compound 25 (150 mg) in N,N-dimethylformamide (4.2 ml). After magnetic stirring for 1 h at 0° C., N-hydroxysuccinimide (58 mg, 0.502 mmol) is added at 0° C., and then, after stirring for 1 h at this temperature, the reaction mixture is left to return to ambient temperature for 1 h. The precipitate is then filtered off (Millipore LS 5 μm), the filtrate is concentrated under a high vacuum, and then the residue is taken up with water, and filtered (Millipore LS 5 μm) to give the desired compound 26 (8.1 mg).

LC-MS m/z 456.1 [(M+H)⁺]. T_{R}=5.355 min

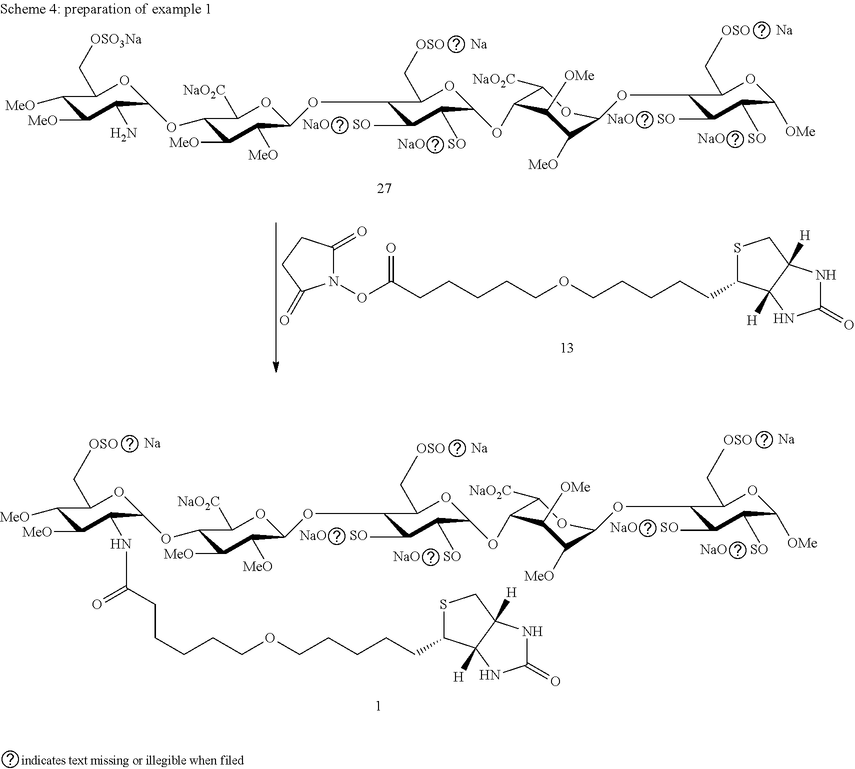

### EXAMPLE 1

### Methyl (2-deoxy-3,4-di-O-methyl-2-{[6-({5-[(3aS,4S,6aR)-2-oxohexahydro-1H-thieno[3,4-d]imidazol-4-yl]penty}oxy)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt (compound No. 1)

A solution of the compound 13 (40.2 mg, 0.091 mmol) in N,N-dimethylformamide (607 μl) is added, at 0° C., to a solution of the compound 27 (52 mg, 0.03 mmol) in an aqueous solution of sodium hydrogen carbonate at 0.5% (607 μl). After stirring for 16 h at ambient temperature, the reaction mixture is loaded onto a column of Sephadex G-25 fine (90×3 cm) eluted with an aqueous 0.2M solution of NaCl. The fractions containing the expected compound are combined and loaded onto a column of Sephadex G-25 fine (90×3 cm) eluted with water. After concentration of the fractions containing the expected compound and lyophilization, 47.8 mg of the compound 1 are obtained.

Chemical shifts of the anomeric protons (600 MHz, D₂O) δ 5.48 Glc^{III}, 5.31 Glc^{V}, 5.21 Glc^{I}, 5.18 IdoUA^{II}, 4.73 GlcUA^{IV}

### [α]_{D} 60° (c 0.99; H₂O)

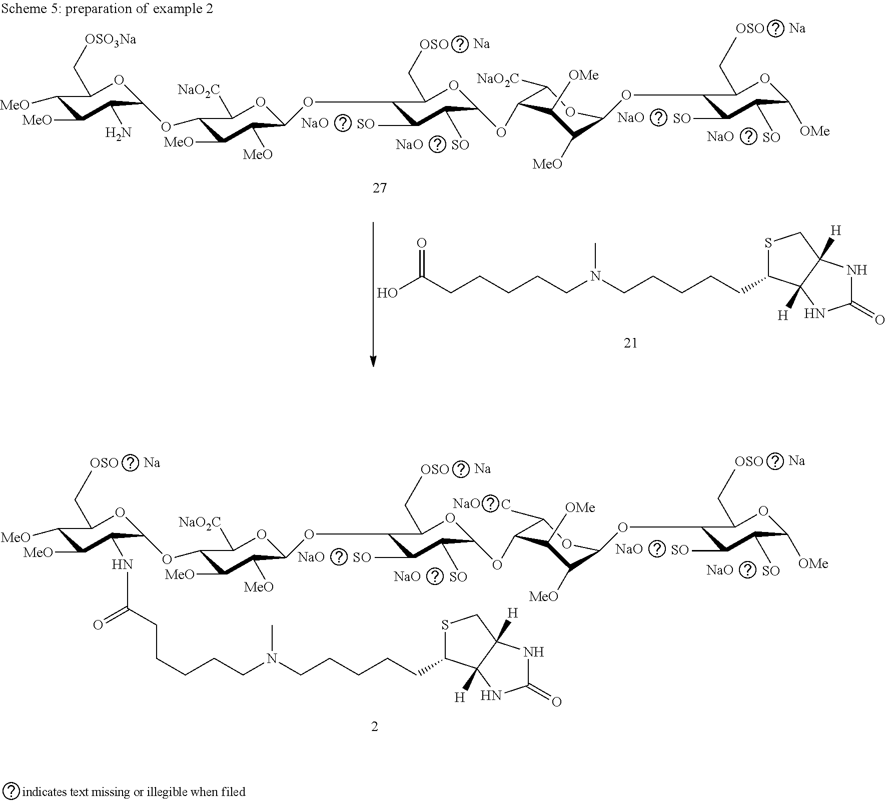

### EXAMPLE 2

### Methyl (2-deoxy-3,4-di-O-methyl-2-{[6-(methyl{5-[(3aS,4S,6aR)-2-oxohexahydro-1H-thieno[3,4-d]imidazol-4-yl]pentyl}amino)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt (compound No. 2)

Triethylamine (23.7 μl, 170 mmol) and ethyl chloroformate (16.3 μl, 170 mmol) are added successively, at −5° C., to a solution of the compound 21 (76 mg, 170 mmol) in N,N-dimethylformamide (1.8 ml). After 1 h, a solution of the compound 27 (180 mg, 85.2 mmol) in N,N-dimethylformamide (1.8 ml) is added to the reaction mixture. After stirring for 2 h at −5° C., the reaction medium is concentrated to dryness. The resulting residue is purified by chromatography on a Carbopac PA100 gel column with a gradient starting from an eluent A (70%) toward an eluent B (53%), the eluent A consisting of a water/acetonitrile (89/11) mixture, and the eluent B consisting of a mixture of a 2N sodium chloride solution and acetonitrile (89/11). The fractions containing the expected compound are combined and loaded onto a column of Sephadex G-25 medium gel eluted with water. After concentration of the fractions containing the expected compound, 32.6 mg of the compound 2 are obtained.

Chemical shifts of the anomeric protons (500 MHz, D₂O) δ 5.42 Glc^{III}, 5.29 Glc^{V}, 5.16 Glc^{I}, 5.08 IdoUA^{III}, 4.71 GlcUA^{IV}

“ESI” method, negative mode: multicharged ion detected m/z 1002.09 [M-2Na]²⁻.

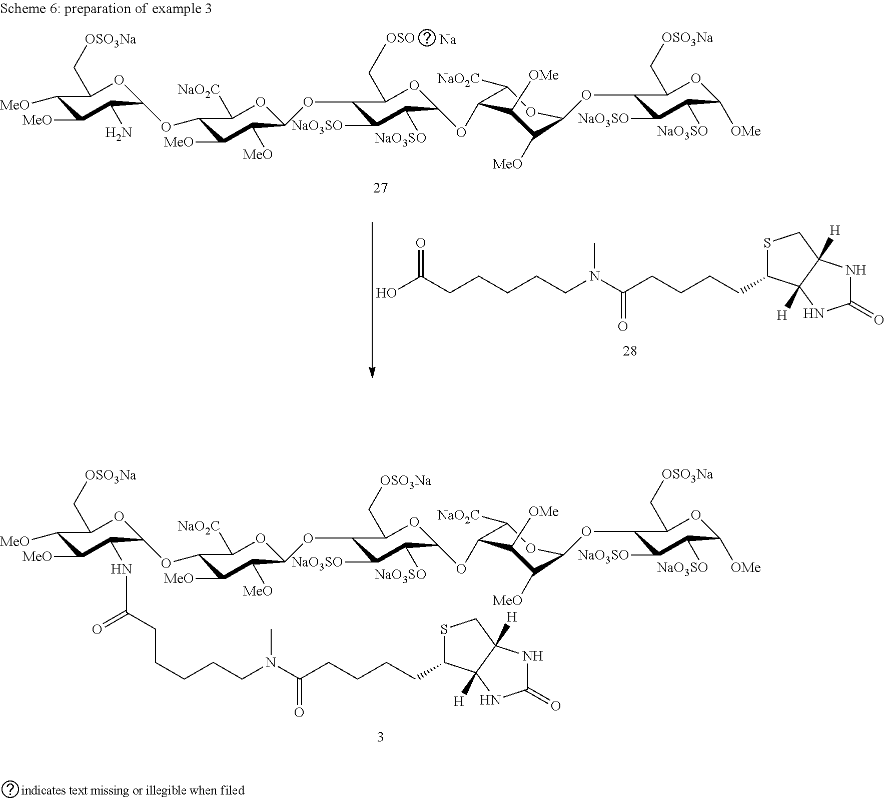

### EXAMPLE 3

### Methyl (2-deoxy-3,4-di-O-methyl-2-{[6-(methyl{5-[(3aS,4S,6aR)-2-oxohexahydro-1H-thieno[3,4-d]imidazol-4-yl]pentanoyl}amino)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-(2,3,6-tri-O-sulfonato-α-D-glucopyranoside, sodium salt (compound No. 3)

Triethylamine (39.5 μl, 283.8 mmol) and ethyl chloroformate (21.7 μl, 283.8 mmol) are successively added, at −5° C., to a solution of 6-(methyl{5-[(3aR,4R,6aS)-2-oxohexahydro-1H-thieno[3,4-d]imidazol-4-yl]pentanoyl}amino)hexanoic acid 28 (105.4 mg, 283.8 mmol; described in patent application WO 97/46098) in N,N-dimethylformamide (3 ml). After 1 h, a solution of the compound 27 (300 mg, 142 mmol) in N,N-dimethylformamide (3.0 ml) is added to the reaction mixture. After stirring for 2 h at −5° C., the reaction medium is concentrated to dryness. The resulting residue is purified by chromatography on a Carbopac PA100 gel column with a gradient starting from an eluent A (55%) toward an eluent B (66%), the eluent A consisting of a water/acetonitrile (89/11) mixture, and the eluent B consisting of a mixture of a 2N sodium chloride solution and acetonitrile (89/11). The fractions containing the expected compound are combined and loaded onto a column of Sephadex G-25 medium gel eluted with water. After concentration of the fractions containing the expected compound, 22.0 mg of the compound 3 are obtained.

Chemical shifts of the anomeric protons (500 MHz, D₂O) δ 5.44 Glc^{III}, 5.26 Glc^{V}, 5.17 Glc^{I}, 5.23 IdoUA^{II}, 4.71 GlcUA^{IV}

“ESI” method, negative mode: multicharged ion detected m/z 1009.0 [M-2Na]²⁻.

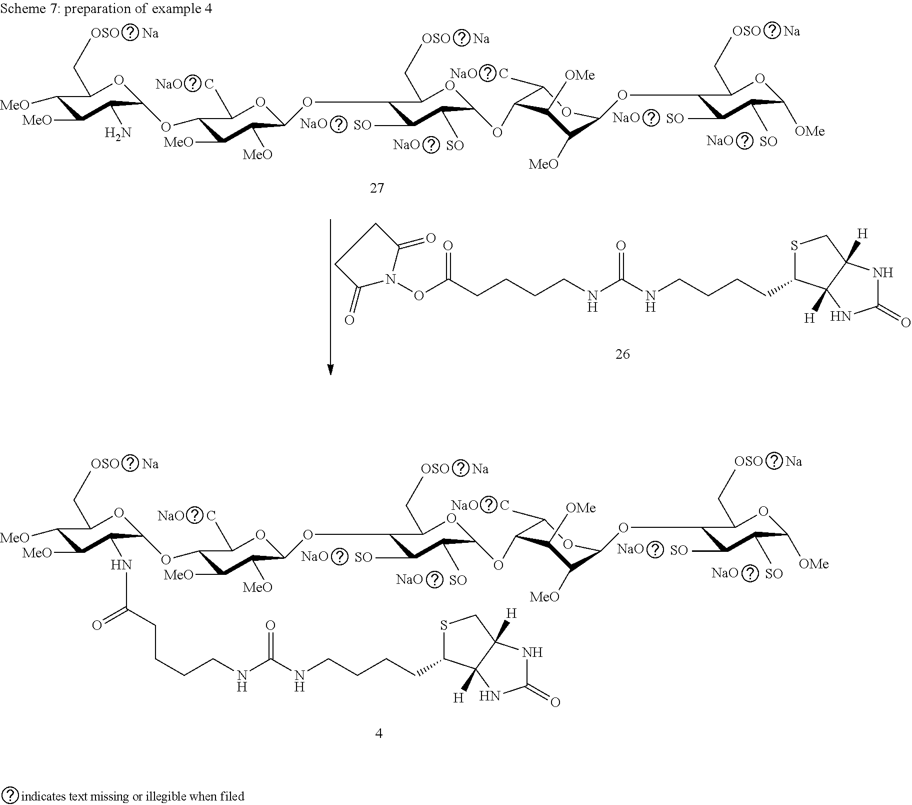

### EXAMPLE 4

### Methyl (2-deoxy-3,4-di-O-methyl-2-({5-[({4-[(3aS,4S,6aR)-2-oxohexahydro-1H-thieno[3,4-d]imidazol-4-yl]butyl}carbamoyl)amino]pentanoyl}amino)-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt (compound No. 4)

A solution of the compound 26 (11.2 mg, 20 μmol) in N,N-dimethylformamide (0.5 ml) is added, dropwise, to an aqueous solution containing 0.5% of sodium chloride (0.5 ml) of the compound 27 (44 mg, 20 μmol). After stirring for 10 min, 0.25 ml of water and also 0.25 ml of N,N-dimethylformamide are added. After vigorous stirring for 16 h at ambient temperature, the reaction medium is loaded at the top of a column of Sephadex® G-25 eluted with an aqueous 0.2M sodium chloride solution. The fractions containing the product are concentrated and desalification is carried out using the same column eluted with water. The fractions containing the product are then concentrated under a high vacuum. The residue obtained is then purified by ion-exchange chromatography using a Dionex CarboPac® PA100 semipreparative column (9×250 mm) with a gradient starting from an eluent A toward an eluent B, the eluent A consisting of a mixture of water/acetonitrile (4/1)+0.01% of dimethyl sulfoxide, and the eluent B consisting of a mixture of a 2N sodium chloride solution and acetonitrile (4/1). The fractions containing the product are then concentrated and desalification is carried out using a Sephadex® G25 column eluted with water. The fractions containing the product are then concentrated under a high vacuum to give the desired compound 4 (18.5 mg, 37%). Chemical shifts of the anomeric protons (600 MHz, D₂O) δ 5.48 Glc^{III}, 5.32 Glc^{V}, 5.22 Glc^{I}, 5.11 IdoUA^{II}, 4.74 GlcUA^{IV}

“ESI” method, negative mode: multicharged ion detected m/z 462.2941 [M-4H]⁴⁻ (acid form).

### Pharmacology:

The compounds according to the invention were the subject of biochemical and pharmacological studies, demonstrating their advantage as compounds of pharmaceutical interest.

Anti-Xa Activity

The activity of the compounds according to the invention was studied in an in vitro model of inhibition of coagulation factor Xa, in the presence of antithrombin (antithrombin-dependent anti-factor Xa activity), as described by J.-M. Herbert et al., Blood, 1998, 91, 4197-4205. In this model, the antithrombotic properties of the compounds according to the invention are confirmed: the 1050 values (doses enabling 50% inhibition) of the compounds No. 1, 2, 3 and 4 are respectively 18.4, 18.4, 17.8 and 17.1 ng/ml.

Thus, the anti-Xa activity of the compounds according to the invention is equivalent to that of the compound idrabiotaparinux for which the IC50 is 17.6 ng/ml.

Neutralization with Avidin

In order to demonstrate that the compounds according to the invention bind well to avidin, the products are diluted, in increasing concentrations, with beads exhibiting avidin molecules at their surface. The resulting mixture is centrifuged and the anti-factor Xa activity is assayed in the supernatant. As it emerges from the table below, the low activity in the supernatant clearly demonstrates that the biotinylated compounds bound to the avidin and were removed by centrifugation with the beads.

| μg product/ | Idrabiota- | | | | |
|---|---|---|---|---|---|
| μg avidin | parinux | Compound 1 | Compound 2 | Compound 3 | Compound 4 |
| 0.01 | 5.2 ± 3.4% | 6.8 ± 2.0% | 4.1 ± 0.2% | 0.9%⁽¹⁾ | 2.4 ± 0.0% |
| 0.001 | 2.6 ± 0.3% | 6.0 ± 0.2% | 5.2 ± 0.5% | 1.8 ± 0.2% | 4.5 ± 0.8% |
| 0.0001 | 2.5 ± 1.1% | 5.6 ± 1.0% | 3.0 ± 0.7% | 1.2 ± 0.6% | 2.5 ± 2.3% |
| 0.00005 | 1.2 ± 0.0% | 1.3 ± 0.9% | 4.9 ± 1.4% | 0.7 ± 2.4% | 3.6 ± 1.2% |

⁽¹⁾Standard deviation not calculated

Thus, more than 90% of the compounds according to the invention are removed from the supernatant by the avidin bound to the beads, in a manner similar to what is observed for the compound idrabiotaparinux.

Metabolic Stability in Plasma Medium at pH 6.0

The metabolic stability of the compounds according to the invention with respect to biotinidase activity was studied on human plasma having been sampled on sodium citrate as anticoagulant. Ten individual batches of human plasma originating from 5 men and 5 women, constituting the evaluation matrix, were used and pooled in order to evaluate the metabolic stability of the compounds. The human plasma was incubated under the following conditions:
incubation medium (freshly prepared): 740 μl of 54 mM of sodium phosphate buffer, pH 6.0, containing 1.08 mM of disodium EDTA and 4.3 mM of cysteamine hydrochloride,
human plasma: 40 μl
concentrations of the compounds to be studied: 1 μM
incubation time: 24 hours
incubation temperature: 37° C.

The reaction is initiated by adding the compound to be studied, after a 10-minute period of preincubation of the plasma and of the incubation medium. At the end of the incubation period (24 hours), the reaction is stopped.

The compounds according to the invention are quantified according to a specific LC/MS-MS method.

The metabolization of each compound studied is measured by monitoring the disappearance of the unchanged product, the concentrations being determined for each incubation sample at time 0 hours and at time 24 hours, and the percentage metabolization being calculated according to the following formula:

$$\% metabolization = [ 1 - ( \frac{[ compound at T 24 H ]}{[ compound at T 0 H ]} ) ] \times 100$$

The terms [compound at T 24H] and [compound at T 0H] correspond to the concentration of the compound to be studied in the reactive medium after 24 hours and before the beginning of the reaction, respectively.

The results are expressed as percentages of the compounds metabolized after 24 h of exposure to the medium. Metabolization percentages of 1.7, −3.3, 0.8 and 5.3%, respectively, are thus measured for the compounds No. 1, 2, 3 and 4 according to the invention, in comparison with a metabolization percentage of 15.9% for the compound idrabiotaparinux.

This demonstrates the metabolic stability of the compounds according to the invention in this in vitro medium: they do not degrade, or they degrade in an extremely low amount, in the presence of biotinidase, unlike idrabiotaparinux.

By virtue of their pharmacological profile, the oligosaccharides of the present invention constitute very advantageous medicaments. Their toxicity is completely compatible with this use. Their metabolic stability with respect to biotinidase makes them particularly suitable for constituting the active ingredient of pharmaceutical specialty products.

They can be used in various pathological conditions subsequent to a modification of the homeostasis of the coagulation system occurring in particular during cardiovascular and cerebrovascular system disorders, for instance thromboembolic disorders associated with artherosclerosis and with diabetes, such as unstable angina, stroke, post-angioplasty restenosis, endarterectomy, the insertion of endovascular prostheses; or thromboembolic disorders associated with post-thrombolysis rethrombosis, with infarction, with dementia of ischemic origin, with peripheral arterial disease, with hemodialysis, with atrial fibrillation or else during the use of vascular prostheses for aortocoronary bypasses. These products can, moreover, be used for the treatment or prevention of thromboembolic pathological conditions of venous origin, such as pulmonary embolisms and deep vein thrombosis. They can be used either for preventing or for treating the thrombotic complications observed, for example, following surgical operations, the growth of tumors or coagulation disturbances induced by bacterial, viral or enzymatic activators. In the case of their use during the insertion of prostheses, the compounds of the present invention can cover prostheses and thus make them hemocompatible. In particular, they can be attached to intravascular prostheses (stents). In this case, they can optionally be chemically modified by introduction, at the nonreducing or reducing end, of an appropriate arm, as described according to EP 649 854. The compounds of the present invention can also be used as adjuvants during endarterectomy carried out with porous balloons.

The compounds according to the invention can be used for the treatment or prevention of the above diseases.

According to another of its aspects, a subject of the present invention is therefore a pharmaceutical composition containing, as active ingredient, a synthetic polysaccharide according to the invention or a pharmaceutically acceptable salt thereof, optionally in combination with one or more suitable inert excipients.

Said excipients are chosen according to the pharmaceutical form and the mode of administration desired: oral, sublingual, subcutaneous, intramuscular, intravenous, transdermal, transmucosal, local or rectal.

The active ingredient can also be provided in the form of a complex with a cyclodextrin, for example α-, β- or γ-cyclodextrin, 2-hydroxypropyl-β-cyclodextrin or methyl-β-cyclodextrin.

The active ingredient can also be released by a balloon containing it or by an endovascular expander introduced into the blood vessels. The pharmacological efficacy of the active ingredient is thus not affected.

In each dosage unit, the active ingredient is present in the amounts suitable for obtaining the desired prophylactic or therapeutic effect. Each dosage unit can contain from 0.1 to 100 mg of active ingredient, preferably from 0.5 to 50 mg and even more preferably from 2.5 to 3.0 mg.

The compounds according to the invention can also be used in combination with one or more other active ingredients that are of use for the desired therapy, such as, for example, antithrombotics, anticoagulants, platelet aggregation inhibitors, for instance dipyridamole, aspirin, ticlopidine or clopidogrel, or glycoprotein IIb/IIIa complex antagonists.

1. A synthetic polysaccharide having antithrombotic activity, comprising at least one covalent bond with biotin or a biotin derivative, wherein said covalent bond is resistant to metabolic cleavage and comprises a linkage X selected from the group consisting of —O—, —N(R)—, —N(R)—CO— and —N(R′)—CO—N(R″)—, wherein R is an alkyl group and R′ and R″, which may be identical or different, are, independently of one another, a hydrogen atom or alkyl group.

2. The polysaccharide according to claim 1, wherein said covalent bond is a linkage of formula T: 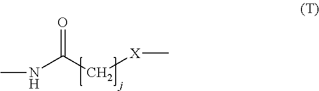
wherein j is an integer from 1 to 10, and X is as defined in claim 1.

3. The polysaccharide according to claim 1, wherein said covalent bond with biotin or the biotin derivative is represented in the formula below: 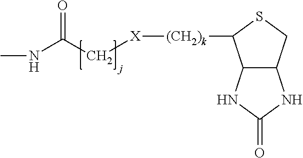
wherein j is an integer from 1 to 10, k is an integer from 4 to 10, and X is as defined in claim 1.

4. The polysaccharide according to claim 1, which is a pentasaccharide.

5. The polysaccharide according to claim 1, comprising the structure of formula (I): 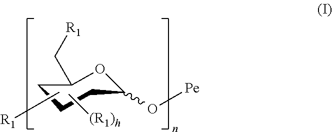
wherein:
the wavy line denotes a bond located either above or below the plane of the pyranose ring,
the formula: 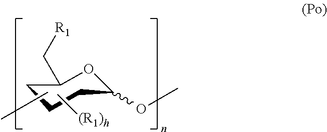
denotes a polysaccharide containing n identical or different monosaccharide units, bonded via its anomeric carbon to Pe,
the formula: 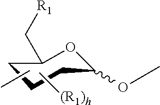
is a diagrammatic representation of a monosaccharide unit with a pyranose structure selected from the group consisting of hexose, pentose, and a corresponding deoxy sugar, wherein the unit is bonded via its anomeric carbon to another monosaccharide unit, and the hydroxyl groups of this unit being substituted with identical or different R₁ groups, and further wherein R₁ is as defined below,
Pe is a pentasaccharide having the structure: 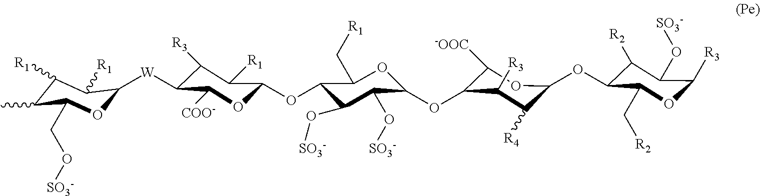
h is equal to 1 or 2,
n is an integer from 0 to 25,
R₁ is the linkage -T-Biot, a (C₁-C₆)alkoxy group or an —OSO₃⁻ group,
R₂ is the linkage -T-Biot, a (C₁-C₆)alkoxy group or an —OSO₃⁻ group,
R₃ is the linkage -T-Biot or a (C₁-C₆)alkoxy group,
R₄ is the linkage -T-Biot, a (C₁-C₆)alkoxy group or an —OSO₃⁻ group, or R₄ constitutes an —O—CH₂— bridge, wherein the —CH₂— group is bonded to the carbon atom bearing the carboxylic function on the same ring;
wherein at least one of R₁, R₂, R₃ or R₄ is the linkage -T-Biot,
W is an oxygen atom or a methylene group,
T is the linkage: 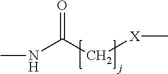
wherein j is an integer from 1 to 10 and X is a linkage selected from the group consisting of —O—, —N(R)—, —N(R)—CO— and —N(R′)—CO—N(R″)—, in which R is an alkyl group and R′ and R″, which may be identical or different, are, independently of one another, a hydrogen atom or alkyl group,
Biot is the group: 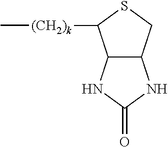
wherein k is an integer from 4 to 10;
or a pharmaceutically acceptable salt thereof.

6. The polysaccharide according to claim 1, comprising the structure of formula (I.1): 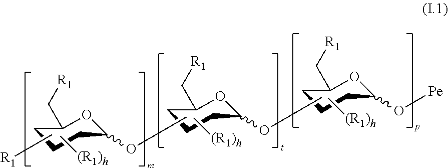
wherein:
the formula: 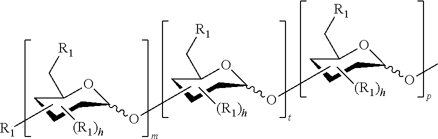
denotes a polysaccharide Po, bonded via an anomeric carbon to Pe as defined for (I),
the formula: 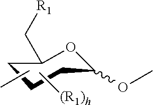
is as defined for (I) as claimed in claim 2,
the R₁ groups are as defined for (I) as claimed in claim 2 and, for one and the same monosaccharide, may be identical or different,
the monosaccharide contained in [ ]ₘ is repeated m times, the monosaccharide contained in [ ]ₜ is repeated t times, and the monosaccharide contained in [ ]ₚ is repeated p times,
m is an integer from 1 to 5, t is an integer from 0 to 24, and p is an integer from 0 to 24, wherein 1≦m+t+p≦25,
or a pharmaceutically acceptable salt thereof.

7. The polysaccharide according to claim 1, comprising a pentasaccharide having the structure of formula (I.2): 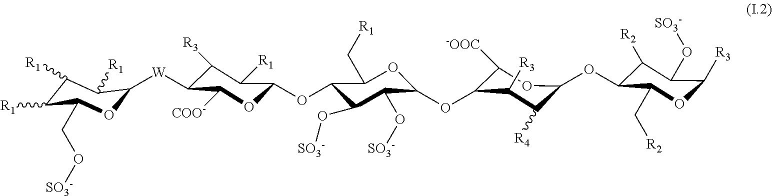
wherein R₁, R₂, R₃, R₄ and W are as defined as claimed in claim 5,
or a pharmaceutically acceptable salt thereof.

8. The polysaccharide according to claim 6, wherein one of R₁, R₂, R₃ or R₄ is the linkage T-Biot with T and Biot being as defined as claimed in claim 5.

9. The pentasaccharide according to claim 7, comprising the structure of formula (I.3): 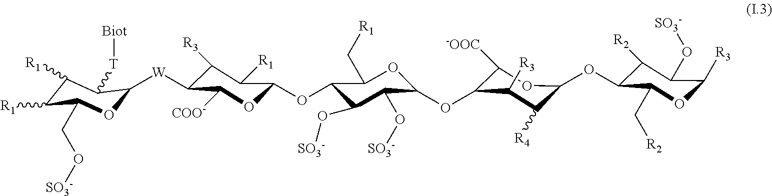
wherein:
T and Biot are as defined in claim 5,
R₁ is a (C₁-C₆)alkoxy group or an —OSO₃⁻ group,
R₂ is a (C₁-C₆)alkoxy group or an —OSO₃⁻ group,
R₃ is a (C₁-C₆)alkoxy group,
R₄ is a (C₁-C₆)alkoxy group or an —OSO₃⁻ group, or else R₄ constitutes an —O—CH₂-bridge, the —CH₂— group being bonded to the carbon atom bearing the carboxylic function on the same ring,
W is an oxygen atom or a methylene group,
or a pharmaceutically acceptable salt thereof.

10. The polysaccharide according to claim 2, wherein j is equal to 4 or 5.

11. The polysaccharide according to claim 3, wherein k is equal to 4 or 5.

12. The polysaccharide according to claim 1, wherein X is a linkage selected from the group consisting of —O—, —N(R)— and —N(R′)—CO—N(R″)—, wherein R and R′ are as defined in claim 2.

13. The polysaccharide according to claim 1, selected from the group consisting of:
Methyl (2-deoxy-3,4-di-O-methyl-2-{[6-({5-[(3aS,4S,6aR)-2-oxohexahydro-1H-thieno[3,4-d]imidazol-4-yl]pentyl}oxy)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt;
Methyl (2-deoxy-3,4-di-O-methyl-2-{[6-(methyl{5-[(3aS,4S,6aR)-2-oxohexa-hydro-1H-thieno[3,4-d]imidazol-4-yl]pentyl}amino)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt;
Methyl (2-deoxy-3,4-di-O-methyl-2-{[6-(methyl{5-[(3aS,4S,6aR)-2-oxo-hexahydro-1H-thieno[3,4-d]imidazol-4-yl]pentanoyl}amino)hexanoyl]amino}-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt; and
Methyl (2-deoxy-3,4-di-O-methyl-2-({5-[({4-[(3aS,4S,6aR)-2-oxohexahydro-1H-thieno[3,4-d]imidazol-4-yl]butyl}carbamoyl)amino]pentanoyl}amino)-6-O-sulfonato-α-D-glucopyranosyl)-(1→4)-(2,3-di-O-methyl-β-D-glucopyranosyluronate)-(1→4)-(2,3,6-tri-O-sulfonato-α-D-glucopyranosyl)-(1→4)-(2,3-di-O-methyl-α-L-idopyranosyluronate)-(1→4)-2,3,6-tri-O-sulfonato-α-D-glucopyranoside, sodium salt.

14. A pharmaceutical composition comprising a polysaccharide according to claim 1 and one or more suitable inert excipients.

15. A method for treating or preventing a pathological condition subsequent to a modification of the homeostasis of the coagulation system occurring during cardiovascular and cerebrovascular system disorders, for instance thromboembolic disorders associated with artherosclerosis or with diabetes, such as unstable angina, stroke, post-angioplasty restenosis, endarterectomy, the insertion of endovascular prostheses, or thromboembolic disorders associated with post-thrombolysis rethrombosis, with infarction, with dementia of ischemic origin, with peripheral arterial disease, with hemodialysis, with atrial fibrillation, during the use of vascular prostheses for aortocoronary bypasses, in the treatment or prevention of thromboembolic pathological conditions of venous origin, such as pulmonary embolisms and deep vein thrombosis, or for preventing or for treating the thrombotic complications observed following surgical operations, the growth of tumors or coagulation disturbances, induced by bacterial, viral or enzymatic activators, the method comprising administering to a subject in need thereof a polysaccharide according to claim 1.

16. The use of a polysaccharide according to claim 1 for covering prostheses or as an adjuvant during an endarterectomy carried out with porous balloons.
